(19) United States
(12) Patent Application Publication
(10) Pub. No.: US 2010/0210943 A1
(43) Pub. Date: Aug. 19, 2010

(54) Systems and Methods for Echoperiodontal Imaging

(75) Inventors: Ahmed M. Mahmoud, Morgantown, WV (US); Osama M. Mukdadi, Morgantown, WV (US); Richard Crout, Morgantown, WV (US); Peter Ngan, Morgantown, WV (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: WEST VIRGINIA UNIVERSITY RESEARCH CORPORATION, Morgantown, WV (US)

(21) Appl. No.: 12/707,973

(22) Filed: Feb. 18, 2010

Related U.S. Application Data
(60) Provisional application No. 61/207,921, filed on Feb. 18, 2009.

(51) Int. Cl. A61B 8/14

(52) U.S. Cl. 600/437

(57) ABSTRACT

Disclosed are various embodiments for echoperiodontal imaging. In one embodiment, a system includes a transducer configured to transmit a series of ultrasonic signals at a plurality of corresponding locations along a jaw and receive a plurality of echo signals; and an imaging system controller configured to obtain a plurality of echo signal data and a plurality of transducer positions, where each echo signal data corresponds to one of the plurality of transducer position. In another embodiment, a method includes transmitting a series of ultrasonic signals at a plurality of corresponding locations along a jaw; receiving a plurality of echo signals; obtaining a plurality of echo signal data and a plurality of corresponding transducer positions; and reconstruct image data of a portion of the jaw for display on a display device based upon the obtained echo signal data and corresponding transducer positions.

### CROSS REFERENCE TO RELATED APPLICATIONS

This application claims priority to copending U.S. provisional application entitled “METHODS AND APPARATUS OF HIGH FREQUENCY ECHODENTOGRAPHIC IMAGING FOR HUMAN JAWBONE ASSESSMENT” having Ser. No. 61/207,921, filed Feb. 18, 2009, which is entirely incorporated herein by reference.

### BACKGROUND

Dental disease adversely affects the teeth and/or tissues that support the teeth of a patient. Periodontal disease, which is an infection of the supporting tissues, is one of the most pervasive dental diseases. The more severe type, periodontitis, can be defined as the presence of gum or gingival inflammation at sites where there has been a pathological detachment of the connective tissue fibers from the cementum or outside covering of the root. In addition, inflammatory events may lead to the resorption or loss of the tooth supporting bone. Imaging of the teeth and the supporting tissues of a patient may assist in diagnosing dental disease.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIGS. 1 and 2 are graphical representations of an exemplary imaging system 100 in accordance with various embodiments of the disclosure.

FIGS. 3 and 4 are graphical representations of an exemplary positioning system of FIG. 1 in accordance with various embodiments of the disclosure.

FIG. 5 is an exemplary embodiment of a transducer cover included in imaging system of FIG. 1 in accordance with various embodiments of the disclosure.

FIG. 6 is a flow chart illustrating the acquisition of echo signal data using the imaging system of FIG. 1 in accordance with various embodiments of the disclosure.

FIG. 7 is a flow chart illustrating the signal processing of echo signal of FIG. 6 and image processing for image reconstruction in accordance with various embodiments of the disclosure.

### DETAILED DESCRIPTION

Disclosed herein are various embodiments of systems and methods related to echoperiodontal imaging. Reference will now be made in detail to the description of the embodiments as illustrated in the drawings, wherein like reference numbers indicate like parts throughout the several views.

Changes that occur in alveolar bone are significant because the destruction of this bone is ultimately responsible for tooth loss. A periodontal defect is defined as an osseous defect in the supporting alveolar bone. Periodontal bony defects can take on many forms. For example, a horizontal defect, often referred to as horizontal bone loss, is the most common pattern of bone loss. Vertical or angular defects are another type of periodontal defect where the bone loss pattern occurs in an oblique direction and leaves a hollowed-out trough in bone adjacent to the tooth root. These defects can also occur on facial or lingual/palatal surfaces of bone supporting a tooth. By this classification, it is possible to have a one-, two- or three-walled defect. Three-walled defects are often referred to as infra-bony defects. Detection and accurate assessment of the location, extent, and configuration of a periodontal defect may assist in determination of a tooth prognosis, treatment plan, and maintenance procedure(s).

When it comes to the detection and diagnosis of periodontal defects, many methods can be used. After the defect(s) are surgical exposured to direct observation, manual probing to discern the borders and dimensions of the defect is the most acceptable clinical assessment of periodontal inflammation. The gold standard for in-depth description of a defect's dimensions is intrasurgical measurement. Only by this method can the clinician see the topography and extent of the defect in its entirety. Obviously, the intrasurgical measurement procedure is the most invasive, costly, and time-consuming. In addition, manual probing is very technique sensitive and thus difficult to standardize between clinicians. Radiography is used in an attempt to supplement the manual probing and provide a “picture” of the defect. Therefore, increasing emphasis is placed on radiography for the detection and description of periodontal defects. Echoperiodontal imaging provides an alternative to both surgical exposure and radiography using ionizing radiation for the detection and diagnosis of periodontal disease.

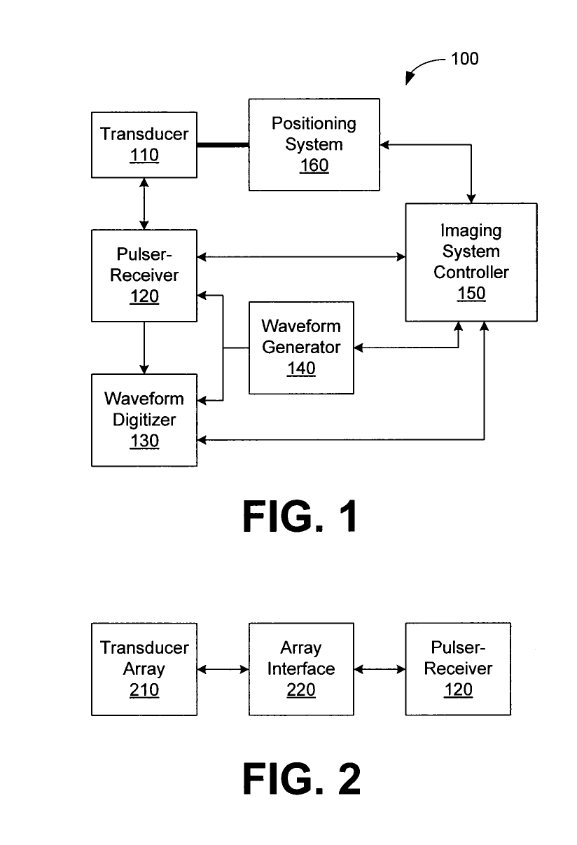

Referring to FIG. 1, shown is a graphical representation of an exemplary imaging system 100 according to various embodiments of the disclosure. The imaging system 100 of FIG. 1 includes an ultrasonic transducer 110, which is used for both transmission of ultrasound signals and reception of the reflected (or echo) signals. In some embodiments, separate transducers may be used for ultrasound transmission and reception. Alternatively, one-dimensional or two-dimensional arrays of transducers 210 (FIG. 2) may be utilized.

The transducer 110 is in communication with a pulser-receiver unit 120. For example, the transducer 110 is connected to a transmit/receive port of the ultrasound pulser-receiver 120. The pulser-receiver 120 may be operated in a pulse-echo mode to provide impulses for use as the excitation signal for the transducer 110. In one embodiment, negative impulses are provided for the excitation signal. The pulser-receiver 120 may include pre-amplification and/or amplification of the excitation signal. For example, the excitation signal may be generated and amplified using a general purpose ultrasonic pulser-receiver such as, but not limited to, an Olympus Model 5900PR pulser-receiver. An exemplary no-load transmission output of the pulser-receiver 120 has an amplitude of 175 Volts with a rise time of two nanoseconds.

The pulser-receiver 120 may provide the excitation signal to stimulate the ultrasound transducers over a wide range of frequencies. For example, frequencies in the range of about 15 MHz to about 100 MHz may be utilized. For high frequency ultrasound, ranges above about 30 MHz may be used. For example, ranges of 30 MHz to about 100 MHz, about 35 MHz to about 100 MHz, and about 50 MHz to about 100 MHz can be used. The frequency range of the excitation signal may be limited by the operational range of the transducer 110 (e.g., a differential single-element ultrasound transducer with a frequency range of 15-30 MHz).

Referring now to FIG. 2, one-dimensional or two-dimensional transducer arrays 210 may be used for transmission of the ultrasound signals and reception of the echo signals. A transducer array 210 may be a linear, curvilinear, or phased array. An array interface 220 may be included between the transducer array 210 and the pulser-receiver 120. The array interface 220 is configured to coordinate application of excitation signals to the transducers of the array 210 and submission of the echo signal received by the transducers of the array 210 to the pulser-receiver 120. The array interface 220 may include a switching matrix that controls the transmit/receive operation and the number of active transducer elements that are working in unison (the group thereby determining the aperture size) and an analog beamforming circuit that controls transmit and receive delays. In one embodiment, the array interface 220 is included in the pulser-receiver 120. In an alternate embodiment, each of a plurality of pulser-receivers 120 may supply a corresponding transducer in the array 120. The plurality of pulser-receivers 120 would then be controlled to coordinate transmission and reception by each transducer.

Referring back to FIG. 1, the echo signal may be amplified and/or filtered by the ultrasonic pulser-receiver 120. The analog echo signal is passed from the pulser-receiver 120 to a high-speed waveform digitizer 130 such as, but not limited to, an analog-to-digital converter (ADC). For example, the echo signal may be digitized and saved with a sampling rate of 62.5 MHz as an 8-bit value using a high-speed data acquisition board. Sampling rates up to 1 GHz may be adopted with higher frequency transducers 110. Similarly, echo signals may be digitized using other bit sizes.

The waveform digitizer 130 is synchronized with the transducer excitation signal to coordinate digital acquisition of the echo signal. In some embodiments, the excitation signal from the pulser-receiver 120 is synchronized with a data acquisition trigger input of the waveform digitizer 130 using a signal generated by a waveform generator 140. An exemplary waveform generator 140 may be, but is not limited to, a computer controlled function generator.

In the embodiment of FIG. 1, an imaging system controller 150 provides system control and synchronization of the pulser-receiver 120, waveform digitizer 130, and/or waveform generator 140 during transmission of the ultrasonic signals and acquisition of the echo signals. In embodiments where a transducer array 210 is utilized, the imaging system controller 150 may also be in communication with the array interface 220 for system control and synchronization. The imaging system controller 150 may be a computer-based system, processor-containing system, or other hardware system that is configured to control and synchronize the imaging system 100.

The imaging system controller 150 of certain embodiments of the present disclosure can be implemented in hardware, software, firmware, or a combination thereof. In the preferred embodiment(s), the imaging system controller 150 is implemented in software or firmware, i.e., instructions that are stored in a memory and that is executed by a suitable instruction execution system. If implemented in hardware, as in an alternative embodiment, the imaging system controller 150 can be implemented with any or a combination of the following technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

In some embodiments, the imaging system 100 also includes a positioning system 160 for movement and positioning of the transducer 110. For example, the positioning system 160 may be a two-axis (providing two degrees of translation) positioning system. In one embodiment, the positioning system 160 is a high-precision positioning system (such as, but not limited to, those produced by Danaher Corp.) with a positioning resolution down to one micrometer. The positioning system 160 includes a positioning controller for a controlling servo amplifier or stepper motor to adjust the positioning of the transducer 110 along each axis. Alternatively, a robotic arm may be used in the positioning system 160. In other embodiments, positioning system 160 provides translation along a single axis.

The imaging system controller 150 synchronizes the positioning system 160 with the data acquisition by the waveform digitizer 130 to collect the ultrasound signals. In some embodiments, the ultrasound signals are collected continuously “on the fly” during the transducer 110 movement. Alternatively, the ultrasound signals may be collected in a step-wise fashion. Transducer 110 position information corresponding to the echo signal data acquisition location may also be collected. In one embodiment, the transducer position is acquired a predetermined time before collecting the echo signal. In other embodiments, the position of the transducer 110 is obtained a predetermined time after transmission of the ultrasonic signal. Alternatively, the transducer position may be the average of the transmission position and acquisition position. In some embodiments, the position is approximated based upon the speed and direction of the transducer motion.

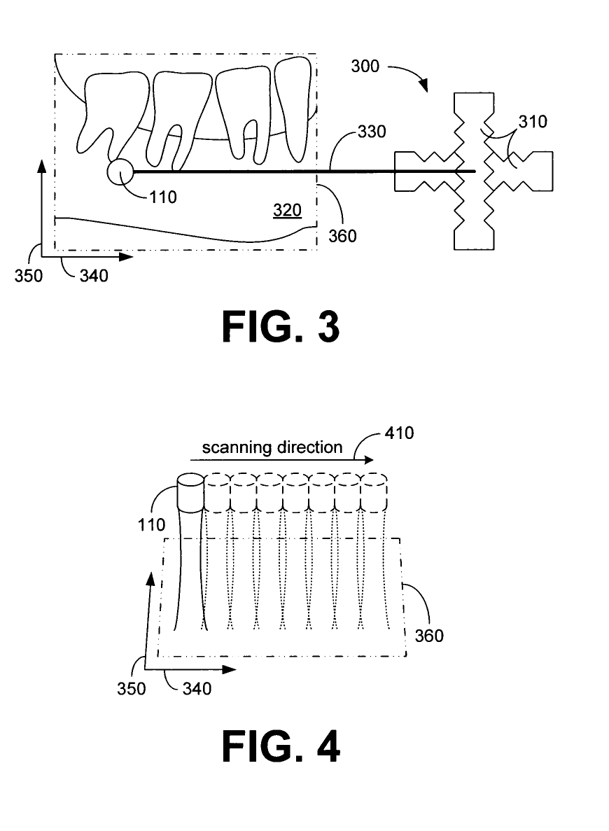

With reference to FIG. 3, shown is a graphical representation of an exemplary two-axis positioning system 300 in accordance with various embodiments of the disclosure. In the embodiment of FIG. 3, two servo amplifiers 310 provide controlled displacement of transducer 110 along a jaw 320 including the teeth, soft tissue, and/or bone that support the teeth. The transducer 110 is supported by an extension arm 330 connected to the positioning system 300. The extension arm 330 may be rigid. Alternatively, the extension arm 330 may be flexible to allow for adjustment of the position and/or orientation of the transducer 110 prior to scanning. The transducer 110 may be coupled to the extension arm 330 by a clamping device, affixed holder, or other appropriate means. Alternatively, the transducer 110 may be integrated into the extension arm 330. The positioning system 300 moves the transducer 110 along orthogonal lateral axis 340 and elevation axis 350. Both spatial position information and echo signal data from a field of view (FOV) 360 of predefined dimensions are collected during movement of the transducer 110 in the FOV 360. The transducer 110 may be oriented at an angle that ranges from 0-90 degrees with respect to the lateral-elevation axis plane.

Referring now to FIG. 4, the positioning system 300 moves the transducer 110 in steps to obtain echo signal data at a series of locations along the lateral axis 340 as illustrated by arrow 410. Alternatively, echo signal data is obtained at the series of locations while the transducer continuously moves along the lateral axis 340. In some embodiments, a one-dimensional lateral scan is performed. In other embodiments, a two-dimensional scan of the FOV 360 may be taken. At the completion of a lateral scan during the two-dimensional scan, the transducer 110 is displaced by a predetermined amount along the elevation axis 350. Echo signal data is then obtained from the transducer 110 as it traverses along the lateral axis 340 at the new elevation. The lateral scan can be in the opposite direction as the previous lateral scan or the transducer 110 can traverse back across the FOV 360 and the scan can be in the same direction as the previous lateral scan. The sequence is repeated until the two-dimensional scan of the FOV 360 is completed. In some embodiments, displacement may be in the range of about 500 μm to about 10 μm. In other embodiments, displacement of the transducer 110 between data acquisition points can be less than 100 μm apart, less than about 50 μm apart, or less than 25 μm apart. For example, acquisition intervals can be in the range of about 50 μm to about 10 μm with data acquisition at intervals of, for example, about 50 μm, about 24 μm, about 15 μm, or about 10 μm.

In other embodiments, the single transducer 110 may be replaced by a one-dimensional or two-dimensional transducer array 210. For example, a row or column of transducers may be attached to the extension arm 330. Data may be sequentially acquired from each transducer in the array, providing echo signal data along the corresponding lateral or elevation axis. The one-dimensional array may then be displaced along the elevation axis or lateral axis, respectively, by a predetermined amount to obtain the next set of echo signal data. If a two-dimensional array is used, data may be sequentially acquired from each transducer in the array without displacement. The transducer array 210 may also be shifted and another set of echo signal data obtained. Thus, allowing for higher resolution data acquisition than that provided by the distribution of the transducers in the array 210.

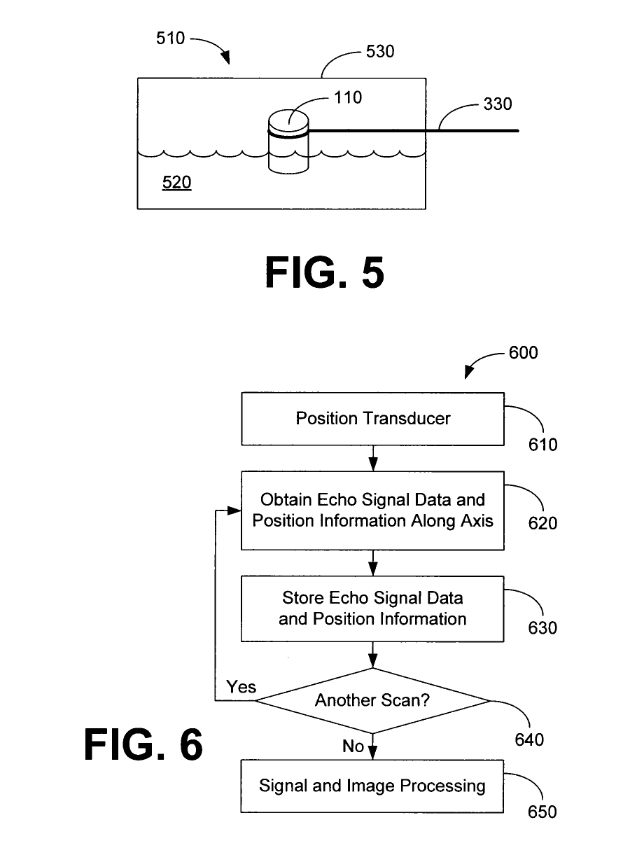

To aid in coupling the ultrasound signals between the transducer 110 and jaw 320, a transducer cover may be utilized. With reference to FIG. 5, shown is an exemplary embodiment of a transducer cover 510. The transducer cover 510 includes a film container 520 that contains a coupling fluid 530 such as, but not limited to, degassed water, and glycol-based, glycerol-based, or water-based liquids and gels. The film container 520 is a flexible material such as, but not limited to, latex, and latex-free polyethylene, and other non-latex materials. The transducer 110 (or transducer array 210) is positioned within the transducer cover 510 (e.g., by extension arm 330) such that the transducer 110 is immersed in the coupling fluid 530. For example, in one embodiment the transducer cover 510 is a bath, which is open at the top, where the transducer 110 extends through the opening into the coupling fluid 530. In this embodiment, the film container 520 of the bath would be supported by a frame (not shown) around the opening. Alternatively, the film container 520 may be a bag or custom designed enclosure that envelops the transducer 110 (or transducer array 210). In this case, the film container 520 is partially or completely filled with the coupling fluid 530 and sealed around the transducer 110 (or transducer array 210) to prevent leakage.

When placed against the jaw 320 and with the transducer 110 immersed in the coupling fluid 530, the transducer cover 510 couples the ultrasonic signals between the transducer 110 and jaw 320. The film container 520 conforms to the surface of the jaw 320. Saliva, water, or oral gel may be applied between the film container 520 and jaw 320 to improve coupling. In some embodiments, the transducer cover 510 is configured to allow repositioning of the transducer 110 (or transducer array 210) within the transducer cover 510 without movement of the transducer cover 510. In other embodiments, the transducer cover 510 is configured to move and/or conform to the surface of the jaw 320 as the transducer 110 is repositioned.

In another embodiment, the transducer 110 may be rotated, without translational movement, in predetermined or measured angles to provide a scan along an axis within the FOV 360. In this case, rotation may be provided through the extension arm 330 by a stepper motor or through linkage to convert linear motion of a servo amplifier to rotation motion. The orientation of the transducer 110 in polar coordinates may be used to reconstruct an image of the jaw 320.

Position information corresponding to each echo signal data acquisition location may also be collected. In one embodiment, the location of the transducer 110 is determined based upon the positioning control by the positioning system 160. A position relative to a starting point may be provided for each acquisition point. Alternatively, magnetic or optical tracking of the transducer position may be used. In this case, a sensor included in the transducer may be used to determine the acquisition position. For example, position and/or orientation may be determined by transmitting magnetic fields with precisely known characteristics. A sensor on the transducer 110 measures the transmitted field and the measurements are used to deduce the sensor (an thus transducer 110) position and/or orientation relative to the transmitter. Alternatively, an optical measurement system can measure the three-dimensional position of active or passive markers affixed to the transducer 110 and thereby determine its position and/or orientation. Where a transducer array 210 is utilized, the transducer location may be predetermined based upon the distribution of transducers within the array 210. Position tracking may also be used with the one-dimensional and two-dimensional transducer arrays 210 to track movement in the lateral or elevation directions.

The imaging system 100 may be programmed with various scanning profiles. For each profile, parameters of the FOV 360 in the lateral and elevation axis such as the dimensions of the FOV 360, as well as the axial (or depth) direction, may be specified. Also, the lateral acquisition speed (frame-rate) that affects distance between ultrasound signals and the elevation step size may be specified. In some implementations (e.g., a 30 mm×30 mm FOV), the scanning process may be completed in less than 30 seconds using a frame-rate of one frame per second (FPS). For a stationary object such as the jaw 320, a frame-rate of one FPS is adequate while providing for patient comfort. In other embodiments, higher frame-rate may be used. After completing the acquisition process, the data is transferred for post processing and image reconstruction.

With reference to FIG. 6, shown is a flow chart 600 illustrating the acquisition of echo signal data using the imaging system 100 in accordance with various embodiments of the disclosure. The transducer 110 (or transducer array 210) is initially positioned in block 610 with respect to the jaw 320 (FIG. 3). In some embodiments, the transducer 110 is positioned at a corner of the FOV 360 where the scan begins. In other embodiments, the transducer 110 is positioned at an initial alignment position (e.g., the center of the FOV 360) and the positioning system 300 (or 160) relocates the transducer 110 to the corner of the FOV 360.

Echo signal data and the corresponding position information are obtained along an axis in block 620. The axis may be in the lateral or elevation direction. The echo signals may be processed (e.g., filtering, amplification, compression, and/or analog beamforming for arrays) before digitizing the signals. After the information is obtained along the axis, the digitized data is stored in memory or on a computer-readable storage medium in block 630. While the exemplary flow chart 600 of FIG. 6 indicates storing the echo signal data and corresponding position information after scanning is complete, in some embodiments echo signal data is stored as it is obtained.

It is then determined in block 640 whether another scan along the axis is to be performed. In the case of a two-dimensional scan with a single transducer 110 or a one-dimensional transducer array 210, the positioning system 300 (or 160) relocates the transducer 110 (or 210) and returns to block 620 to obtain the echo signal data and the corresponding position information along the newly offset axis (next row or column) within the FOV 360. If a two-dimensional transducer array 210 is used, the next set of information along the axis may be obtained without relocation of the transducer array.

While the exemplary imaging system 100 of FIG. 1 includes a positioning system 160 that controls the transducer 110 location during scanning, a hand-held transducer 110 or transducer array 210 with manual positioning may alternatively be used in the imaging system 100. For example, a two-dimensional transducer array 210 may be manually positioned over a desired FOV 360 and the acquisition of echo signal data may proceed as described by flow graph 600 of FIG. 6. Transducer position information may be obtained using magnetic or optical tracking of the position and/or orientation of the transducer array 210. In some embodiments, a plurality of array scans may be combined for a FOV 360 that is larger than the size of the transducer array 210.

When the ultrasonic scan has been completed for the FOV 360 or if only a one-dimensional scan is to be performed, the stored echo signal data and corresponding position information may then be processed and used for image processing in block 650. To this end, the imaging system controller 150 of the imaging system 100 of FIG. 1 may provide signal processing of the acquired echo signal data. Alternatively, a separate computer may provide the signal processing.

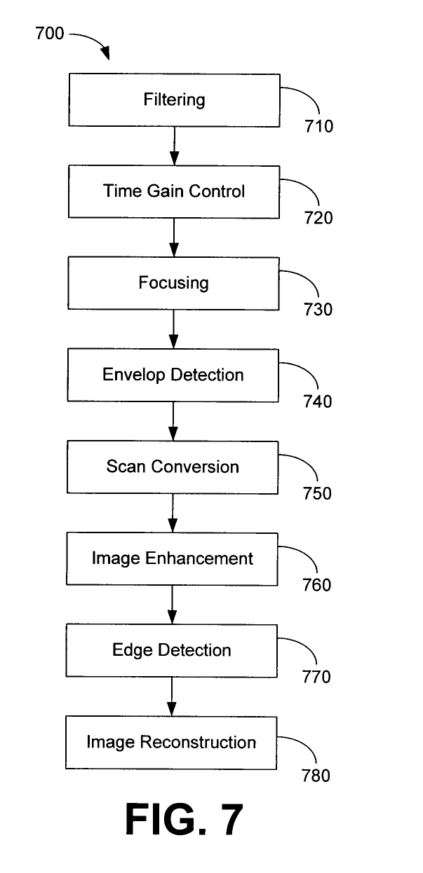

With reference to FIG. 7, shown is a flow chart 700 illustrating the signal processing of the echo signal and image processing for reconstruction of a two-dimensional or three-dimensional image of the jaw 320. The acquired echo signals are first filtered in block 710. Filtering may be completed using a band-pass filter with a pass-band to remove the noise from the acquired data. Exemplary pass-bands include about ±10%, about ±20%, about ±25%, or about ±50% of the transducer center frequency. In block 720, time-gain control (TGC) is applied to compensate for the attenuation effect. In come embodiments, TCG may be accomplished using simple linear amplification. In some embodiments, nonlinear amplification may be used. Alternatively, analog or digital methods using look-up tables may be used for amplification. Generally, TCG amplifies the signal based upon the arrival time, e.g., later signals undergo greater amplification to correct signal amplitude loss.

A focusing procedure is then applied in block 730. A weighted synthetic aperture focusing technique (SAFT), which has been widely used for single-element systems can be used for focusing. In one embodiment, the focusing techniques such as those described by “Synthetic aperture techniques with a virtual source element” by Frazier, C H and O'Brien, W. D. in IEEE Transactions on Ultrasonics Ferroelectrics and Frequency Control, vol. 45, pp. 196-207 (1998) and “Improved synthetic aperture focusing technique with applications in high-frequency ultrasound imaging” by Li M. L., Guan W. J., and Li, P. C. in IEEE Transactions on Ultrasonics Ferroelectrics and Frequency Control, vol. 51, pp. 63-70 (2004), both of which are hereby incorporated by reference in their entireties, can be used. These focusing techniques may be adapted to assure homogenous focusing at various depths.

Also, a combination of conventional B-mode and synthetic aperture imaging techniques may overcome the limited depth of field for a highly focused transducer. Moreover, lateral resolution beyond the transducer focus may be improved by considering the focus a virtual element and applying synthetic aperture focusing techniques. In some embodiments, a boxcar apodization window is used with SAFT for lowering the sidelobes, since it produces images with improved lateral and axial resolution at all depths (see e.g., “High frequency precise ultrasound imaging system to assess mouse hearts and blood vessels” by Mahmoud, A. M., Cortes, D. H., Mustafa, S. J., and Mukdadi, O. M. in Proceedings of the ASME Summer Bioengineering Conference, Marco Island, Fla. (June 2008), which is hereby incorporated by reference in its entirety).

After applying the focusing procedure, a signal processing algorithm is applied in block 740 to detect and extract the signal envelope. For example, calculating the amplitude of the Hilbert transform of the echo signal may be used for the envelope detection. While the signal processing of blocks 710-740 is described as being applied to the digitized echo signal data, in alternate embodiments some or all of the signal processing of blocks 710-740 (such as, but not limited to, filtering, amplification, TGC, Hilbert, and/or compression) may be applied to the echo signal in the analog domain before digitization.

Scan conversion is then accomplished in block 750. Logarithmic compression can be applied to reduce the dynamic range for visualization. Rejection may also be applied here, where a threshold is placed on the signals to reject pulses with amplitudes below the threshold and thus reduce noise content. At this point, a B-mode image is obtained.

Image enhancement techniques may be applied to the B-image in block 760 to reveal small details and to improve the contrast of the converted image. In some embodiments, image smoothing is accomplished. Smoothing and speckle denoising may be applied using, for example, a Perona-Malik algorithm. Since the Perona-Malik algorithm encourages smoothing within homogeneous regions and discourages smoothing between homogeneous regions, the resulting images show a better edge contrast than those obtained using Gaussian convolution. A phase-preserving algorithm based on decomposing the signal using complex-valued wavelets may also be employed to improve the image quality and decrease the noise (see e.g., “Phase preserving denoising of images” by Kovesi, P. in Proceedings of the Australian Pattern Recognition Society Conference: DICTA '99 Perth, Wash., pp. 212-17 (1999), which is hereby incorporated by reference in its entirety).

In addition, non-orthogonal complex valued log-Gabor wavelets may be used to convert the image to the transform domain in block 760. This can be done by applying discrete wavelet transform using wavelets based on complex valued Gabor functions, sine and cosine waves, each modulated by a Gaussian function. Using two filters in quadrature enables the evaluation of the amplitude and the phase of the signal for a particular frequency at a given spatial location. Log-Gabor filters allow arbitrarily large bandwidth filters to be constructed while still maintaining a zero DC component in the even-symmetric filter. Moreover, these filters help to minimize the spatial spread of wavelet response to signal features, and hence concentrate the signal energy into a limited number of coefficients. In some embodiments, appropriate wavelet shrinkage thresholds are automatically determined from the statistics of the amplitude response of the smallest scale wavelet quadrature pair. Transforms are then clipped at the threshold and the inverse transform is taken for optimal image improvement. The images may then be linearly mapped to gray scale levels for display at the proper dynamic range (e.g., 80 dB).

In some embodiments, a high quality and high resolution two-dimensional B-mode image of the jaw may be obtained from the enhanced image. This B-mode image may include details regarding both soft tissue and bone of the jaw. Three-dimensional volume images may also be produced using a plurality of two-dimensional B-mode images.

Edge detection is then performed in block 770. In the processed B-mode images, the outer boundary of the bone appears brighter than any other region. This brightness results from the high reflection coefficient in the tissue-bone interface that causes strong reflections. The bone boundary for each image may be detected by applying a slight image thresholding procedure, followed by edge detection algorithm. In some embodiments, the bone surface may be approximated as the location where the first maximum reflection occurs. Alternatively, the surface may be approximated using a percentage of the maximum reflection (e.g., 10%). In other embodiments, the bone surface is approximated as the location where the second maximum reflection occurs to take into account a first maximum refection produced by the soft tissue (or coupling interface). The arrival time of the boundary (soft tissue and/or bone surface) for each image line is recorded. Arrival times may be saved in a two-dimensional matrix as a row for each image boundary. For example, each row may represent a lateral scanning and each column may represent the elevation direction. By assuming a homogenous speed of sound (e.g., 1540 m/s) the bone and/or soft tissue surface distance can be determined.

A two-dimensional (e.g., depth and width along either lateral or elevation axis) or three-dimensional (e.g., depth and width along both lateral and elevation axis) surface image is then reconstructed in block 770. Image data used to display the image on a display device is determined by plotting the recorded arrival times, which represent the bone and/or soft tissue boundary, within the ultrasound scanning area. A cubic smoothing spline function may be utilized to smooth the two-dimensional or 3-dimensional mesh and to interpolate missing regions, before displaying the two-dimensional or three-dimensional surface image of the soft tissue and/or bone surface of the jaw. In some embodiments, information for the three-dimensional image is used to fabricate three-dimensional models of the bone surface using a three-dimensional printer. Image resolution of less than 100 μm, less than about 50 μm, or less than 25 μm can be achieved. For example, resolutions of about 50 μm, about 24 μm, about 15 μm, or about 10 μm may be provided. The axial (depth) and lateral resolutions can be described by:

$${R}_{iat} = λ * \frac{FD}{A} and {R}_{ax} = \frac{1}{2} * \frac{c}{BW} .$$

where λ is the wavelength at the transducer center frequency calculated using a speed of 1540 m/s, FD the focal distance, A the transducer diameter, c the speed of sound, and BW the transducer bandwidth.

The flow charts of FIGS. 6 and 7 show the architecture, functionality, and operation of a possible implementation of the exemplary imaging system 100 of FIG. 1. In this regard, each block represents a module, segment, or portion of code, which comprises one or more executable instructions for implementing the specified logical function(s). It should also be noted that in some alternative implementations, the functions noted in the blocks may occur out of the order noted in FIGS. 6 and 7. For example, two blocks shown in succession in FIGS. 6 and 7 may in fact be executed substantially concurrently or the blocks may sometimes be executed in the reverse order, depending upon the functionality involved.

Having described the operation and structure of the imaging system 100, experimental results will now be discussed. A first experimental evaluation was carried out on a dry mandible. Twenty-four lateral scans (or frames) were acquired for a region of the mandible. The mandible included landmarks describing (1A) a horizontal bony defect between two premolars, (2A) a distinct image of the mental foramen, and (3A) a severe vertical bony defect adjacent to the distal root of a first molar. For each frame, the echo signals were collected 45.5 μm apart to cover a scan (or FOV) width of 25.9 mm. The mandible was placed in degassed water near the transducer focus with an angle of 30 degrees. This angle can be changed according to the location of the region of interest within the mandible. Frames were acquired 0.5 mm apart with respect to the transducer elevation axis, starting near the tooth roots. A three-dimensional surface image of the FOV was generated based upon the obtained echo signal data and corresponding position information. Using the techniques described herein, the topography of the three landmarks in the image matched the anatomy of the mandible. In contrast, an x-ray image of the FOV does not include landmarks (2A) and (3A), which may be due to the projection of the x-ray image. Moreover, while landmark (1A) can be identified from the x-ray image, it provides no information about the type of periodontal defect in the FOV.

In another evaluation, a thin slice of meat was attached to a mandible with a severe three-wall bony defect around the second molar to simulate the soft tissue of the jaw. The mandible included landmarks describing (1B) the root of a second molar, (2B) the bone line after the pocket of the defect, and (3B) the bone edge at the end of the defect, which exists between the three landmarks. Ultrasound scanning was performed, for a FOV around the defect on the mandible after attaching the tissue. Sixteen lateral scans were acquired 0.5 mm apart in the elevation axis, while the echo signals were collected 44 μm apart in the lateral direction. The experimental setup was similar to the previous experiment for dry mandible. The signal processing procedures utilized a thresholding algorithm to detect the bone surface under the tissue. The three-dimensional ultrasound surface image of the jawbone produced by the techniques described herein described all the three landmarks. Moreover, the three-wall defect can be identified quantitatively in the three directions (lateral, elevation, and axial). In a corresponding x-ray image for the same site of the mandible, landmarks (1B) and (2B) are almost projected into the same point, reducing the ability to distinguish the extent of the defects. Moreover, it was difficult to extract information about the pocket end using landmark (3B).

It should be noted that numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, at least the following is claimed:

1. A system for echoperiodontal imaging, comprising:
a transducer positioned adjacent to a jaw, the transducer configured to:
transmit a series of ultrasonic signals at a plurality of corresponding locations along the jaw, and
receive a plurality of echo signals, each echo signal corresponding to one of the plurality of transmitted ultrasonic signals; and
an imaging system controller configured to:
coordinate transmission of the series of ultrasonic signals and reception of the corresponding echo signals, and
obtain a plurality of echo signal data and a plurality of transducer positions, where each echo signal data corresponds to one of the plurality of transducer positions, and where each of the plurality of received echo signals corresponds to one of the plurality of echo signal data and the one corresponding transducer position.

2. The system of claim 1, wherein the plurality of corresponding locations are distributed along a linear path by a predetermined distance.

3. The system of claim 2, wherein the predetermined distance is less than 100 micrometers.

4. The system of claim 3, wherein the predetermined distance is in the range of about 50 micrometers to about 10 micrometers.

5. The system of claim 2, wherein the transducer is further configured to:
transmit a second series of ultrasonic signals at a plurality of corresponding locations distributed along a second linear path by the predetermined distance, the second linear path in parallel with the first linear path, and
receive a second plurality of echo signals, each echo signal corresponding to one of the second plurality of transmitted ultrasonic signals along the second linear path; and
wherein the imaging system controller is further configured to obtain a second plurality of echo signal data and a second plurality of corresponding transducer positions, where each of the second plurality of echo signal data corresponds to one of the second plurality of transducer position, and where each of the second plurality of received echo signals corresponds to one of the second plurality of echo signal data and the one corresponding transducer position.

6. The system of claim 1, wherein the imaging system controller is further configured to reconstruct an image of a portion of the jaw based upon the obtained echo signal data and corresponding transducer positions.

7. The system of claim 6, wherein the image is a three-dimensional surface image of at least a portion of a jawbone of the jaw.

8. The system of claim 6, wherein resolution of the image is less than 100 micrometers.

9. The system of claim 8, wherein resolution of the image is in the range of about 50 micrometers to about 10 micrometers.

10. The system of claim 1, further comprising a positioning system coupled to the transducer, the positioning system configured to move the transducer between the plurality of corresponding locations, wherein the imaging system controller is further configured to coordinate movement of the transducer by the positioning system with the transmission of the series of ultrasonic signals and the reception of the corresponding echo signals.

11. The system of claim 10, wherein the positioning system continuously moves the transducer across the plurality of corresponding locations.

12. The system of claim 1, further comprising a transducer cover including a film container and a coupling fluid within the film container, the transducer positioned within the transducer cover and immersed in the coupling fluid.

13. The system of claim 12, wherein the coupling fluid is degassed water.

14. The system of claim 1, wherein the transducer is a transducer array.

15. The system of claim 14, wherein the imaging system controller is configured to obtain the plurality of echo signal data and the plurality of corresponding transducer positions along a first row of the transducer array.

16. The system of claim 15, wherein the imaging system controller is further configured to obtain a second plurality of echo signal data and a second plurality of corresponding transducer positions along a second row of the transducer array.

17. The system of claim 1, further comprising a waveform digitizer configured to digitize each received echo signal to produce the echo signal data.

18. A method for echoperiodontal imaging, comprising:
positioning a transducer adjacent to a jaw;
transmitting a series of ultrasonic signals at a plurality of corresponding locations along the jaw;
receiving a plurality of echo signals, each echo signal corresponding to one of the plurality of transmitted ultrasonic signals;
obtaining a plurality of echo signal data and a plurality of corresponding transducer positions, where each echo signal data corresponds to one of the plurality of transducer position, and where each of the plurality of received echo signals corresponds to one of the plurality of echo signal data and the one corresponding transducer position; and
reconstructing image data of a portion of the jaw for display on a display device based upon the obtained echo signal data and corresponding transducer positions.

19. The method of claim 18, wherein the series of ultrasonic signals are transmitted at a plurality of corresponding locations along a linear path.

20. The method of claim 19, wherein the transducer is a one-dimensional transducer array.

21. The method of claim 19, further comprising:
transmitting a second series of ultrasonic signals at a plurality of corresponding locations along a second linear path in parallel with the first linear path;
receiving a second plurality of echo signals, each echo signal corresponding to one of the second plurality of transmitted ultrasonic signals; and
obtaining a second plurality of echo signal data and a second plurality of corresponding transducer positions, where each of the second plurality of echo signal data corresponds to one of the second plurality of transducer position, and where each of the second plurality of received echo signals corresponds to one of the second plurality of echo signal data and the one corresponding transducer position.

22. The method of claim 21, wherein the transducer is a two-dimensional transducer array.

23. The method of claim 18, further comprising providing the image data for display of a three-dimensional surface image of a portion of at least a jawbone of the jaw on a display device.
